(19) United States
(12) Patent Application Publication
(10) Pub. No.: US 2021/0183513 A1
(43) Pub. Date: Jun. 17, 2021

(54) METHOD AND SYSTEM FOR DISEASE PREDICTION AND CONTROL

(71) Applicant: National Chiao Tung University, Hsinchu City (TW)

(72) Inventors: Wen-Liang Chen, Hsinchu (TW); Hsiao-Ching Lee, Hsinchu (TW); Chia-Heng Lin, Hsinchu (TW); Cheng-Hung Wu, Hsinchu (TW); Chun-Wei Liang, Hsinchu (TW); Tzu-Hsuan Lin, Hsinchu (TW); Tiffany Huang, Hsinchu (TW); Yi-Ting Chou, Hsinchu (TW); Ferng-Chang Chang, Hsinchu (TW); Peng-Tzu Chen, Hsinchu (TW); Chia-Hsuan Lin, Hsinchu (TW); Jung-Yu Liu, Hsinchu (TW); Chen-Chuan Wu, Hsinchu (TW); Tien-Yu Chang, Hsinchu (TW); Yu-Chiao Lo, Hsinchu (TW); Kai-Hsiang Su, Hsinchu (TW); Ying-Xin Li, Hsinchu (TW); Ming-Jie Guo, Hsinchu (TW)

(21) Appl. No.: 16/759,186

(22) PCT Filed: Oct. 29, 2018
(86) PCT No.: PCT/MY2018/000033

§ 371 Date: Apr. 24, 2020

Related U.S. Application Data
(60) Provisional application No. 62/577,764, filed on Oct. 27, 2017.

(51) Int. Cl. G16H 50/20; G16H 40/67; G16H 50/70; G06N 3/04; G06N 3/08; G06N 3/12

(52) U.S. Cl. CPC G16H 50/20; G16H 40/67; G06N 3/126; G06N 3/0454; G06N 3/08; G16H 50/70

(57) ABSTRACT

The present disclosure provides a method for disease control of plants, comprising predicting the probability of a disease occurrence and suggesting a suitable and effective control measure for the identified pathogen and/or host. The present disclosure also provides an advisory service with recommended management actions and other alerts and notifications.

### TECHNICAL FIELD

The present disclosure relates generally to disease prediction and control, and relates particularly to a method for predicting the occurrence of a disease and controlling the disease with a predicted treatment for the disease.

### BACKGROUND

Plant diseases caused by pathogens such as fungi affect crops and soils of farms and are the constant problems for the agricultural industry. Fungal diseases could account for as many as two-thirds of the total plant diseases. Frequently, chemical pesticides are applied, or the entire farmland is abandoned to eliminate fungal diseases. Plant diseases thus lead to huge economic losses. The improvement and application of pest and disease models to predict the occurrence of pathogen infection and to suggest proper control measures are still a challenge for the agricultural community, especially with the extent of the climate change observed in the recent years.

However, the leaping advance of information technology in the recent decades provides an innovative approach to solve problems in different fields, including agriculture. Modeling of crop diseases and pests targeted at the development of support capabilities to schedule scouting or pesticide applications has been applied. A better prediction for the time and probability of disease occurrence, or even the type of the pathogen and suitable pesticide is favorably desired.

On the other hand, while various types and great amount of information related to crop management could be collected and stored for later analysis now, there is a need for efficient utilization of the vast data. Organizing and assessing such amount of data and finding the best approach for analysis can be time-consuming, and there is a need for efficient modeling of data that could process vast data in a short time to allow timely action in response to the pathogenic threats.

### SUMMARY

The present disclosure is to provide a system for disease control of plants. Also, the present disclosure is to predict probability of a disease occurrence, and recommend a suitable and effective control measure for an identified pathogen and/or crop. The present disclosure further provides an integrated database that includes related data for the prediction of the effective control measure for plant diseases. Also provided in the present disclosure is a system and method of examining weather conditions and crop management practices to model a risk of disease occurrence in a field over a specific time period, and generate a prediction of the disease occurrence in the field. Still provided in the present disclosure is an indication to growers, landowners, crop advisors, and other responsible entities of a possible pathogen presence in a field under observation to enable one or more responsive management actions. Yet still provided in the present disclosure is an advisory service with recommended management actions and other alerts and notifications to such growers, landowners, crop advisors and other responsible entities where this is a risk or prediction of pathogen presence in a field under observation.

The present disclosure provides a system for disease control, comprising: a plurality of sensors configured to detect environmental information; and a processor configured to build up a disease prediction model by collecting disease data and weather data, combining the disease data and the weather data to form combined data, processing the combined data by a machine training and testing process, and identifying a plurality of patterns of disease occurrence, wherein the disease prediction model is configured to calculate a probability of the disease occurrence according to the environmental information and the patterns. In one embodiment, the weather data collected by the disease prediction model includes at least one of observation time, pressure, temperature, dew point temperature, relative humidity, wind speed, wind direction, precipitation, sunshine duration, visibility, ultraviolet index, and cloud amount.

In one embodiment, the disease data of the disease prediction model includes a positive label and a negative label indicating the disease occurrence.

In one embodiment, the processor of the disease prediction model is configured further by extracting features from the disease data and the weather data, wherein the features are processed by the processor for the machine training and testing process.

In one embodiment, the machine training and testing process associates with Convolutional Neural Network (CNN).

In one embodiment, the present disclosure provides a system for disease control with the sensors configured to send the environmental information to the disease prediction model through an Internet of Thing (IoT) technology. In another embodiment, the environmental information includes at least one of relative humidity, temperature, rainfall, and pressure. In yet another embodiment, the weather data are collected over a period of time for 5 days, 7 days, 10 days, 14 days, 18 days or 21 days. In an embodiment, the weather data are collected over a period of time for 14 days.

In one embodiment, the present disclosure also provides a system for disease control, wherein the processor is configured to build up the disease prediction model further by classifying the patterns into a negative output indicating a disease not to be happened or a positive output indicating a disease to be happened. In another embodiment, the disease prediction model is further configured to raise warning according to the negative output or the positive output. In another embodiment, the processor is further configured to build up a spore generation model configured to calculate a spore generation rate based on the environmental information. In yet another embodiment, the spore generation model is based on relative humidity and temperature. In still yet another embodiment, the relative humidity and the temperature upon which the spore generation model is based on are independent events.

In one embodiment, the present disclosure provides a system for disease control, wherein the processor is configured to provide the time of the disease occurrence through the disease prediction model and the spore generation model. In another embodiment, the disease prediction model or the spore generation model is configured to send the probability of the disease occurrence or the time of the disease occurrence to a spraying system through an Internet of Thing (IoT) technology. In yet another embodiment, the spore generation rate is Botrytis cinerea's spore germination rate, Myce-liophthora thermophila's spore germination rate, Aspergillus niger's spore germination rate, P. oryzae's spore germination rate, Diplodia corticola's spore germination rate, or Pseudocercospora's spore germination rate.

In one embodiment, the present disclosure also provides a system for disease control, wherein the processor further includes a peptide prediction model configured to predict a peptide with an antifungal function by a Scoring Card Method (SCM). In another embodiment, the peptide prediction model involves calculating a score for a peptide by determining the propensities of dipeptides that make up the peptide. In yet another embodiment, the peptide prediction model involves calculating a score for a peptide by analysis of sequence of the peptide. In another embodiment, the peptide prediction model is further configured to comprise a search system containing relationships of hosts, pathogens, and corresponding peptides. In a further embodiment, the system for disease control is connected to a spraying system configured to spray the peptide with the antifungal function on a field based on the probability of the disease occurrence.

Other embodiments and features of the present disclosure will become apparent from the following descriptions of the embodiments, taken together with the accompanying drawings, which illustrate, by way of example, the principles of the disclosure.

### BRIEF DESCRIPTION OF THE DRAWINGS

FIG. 1 shows the illustration of a peptide displayed as a group of dipeptides.

FIG. 2 shows the number of the datasets collected and used in the peptide prediction model.

FIG. 3 illustrates the procedures to calculate a peptide score by the score card.

FIG. 4 shows the flow chart of IGA implementation.

FIG. 5 shows the four classes used in the confusion matrix for fitness calculation.

FIG. 6 shows the ROC curve drawn taking TPR as the y-axis and FPR as x-axis for fitness calculation.

FIG. 7 shows the separating of each weight of the score card into different areas in proportion to its fitness as used in the roulette method.

FIG. 8 shows the procedures of crossover in IGA.

FIG. 9 shows how the parameters are determined for crossover.

FIG. 10 shows a final ROC curve and the result of test datasets with an antifungal peptide having sequence identity of 25% according to the antifungal peptide prediction.

FIG. 11 shows the score distributions of the positive datasets and the negative datasets with an antifungal peptide having sequence identity of 25% according to the antifungal peptide prediction.

FIG. 12 shows the final antifungal scoring card of the dipeptide scores.

FIG. 13 shows the bar graphs of the single amino acid score calculated from each dipeptide score.

FIG. 14 shows the shaded 3D structure of Rs-AFP2 according to the dipeptide scores calculated by the prediction model.

FIG. 15 shows the 3D structure of Rs-AFP2 peptide with its active region shaded darker according to the report in the literature.

FIG. 16 shows the flow chart of data processing used in the disease prediction model.

FIG. 17 shows an overview of the CNN method used in the disease prediction model, where it contains the convolution layer, max pooling layer and multi full connection layer.

FIG. 18 shows the flow chart to improve the accuracy of the disease prediction model.

FIG. 19 shows the result of the independent test data for the disease prediction model.

FIG. 20 shows Myce-liophthora thermophila's spore germination rate based on temperature.

FIG. 21 shows Aspergillus niger's spore germination rate based on temperature.

FIG. 22 shows P. oryzae's spore germination rate based on temperature.

FIG. 23 shows Diplodia corticola's spore germination rate based on temperature.

FIG. 24 shows Aspergillus niger's spore germination rate based on relative humidity.

FIG. 25 shows Pseudocercospora's spore germination rate based on relative humidity.

FIG. 26 shows the experiment design to determine the coefficients for the general fungal spore germination model and to verify the model.

FIG. 27 shows the photo of the spores that had not germinated at 10 degree Celsius and 100% relative humidity for 9 hours.

FIG. 28 shows the photo of the germinated spores at 25 degrees Celsius and 100% relative humidity for 9 hours.

FIG. 29 shows the table of the spore germination rates of Botrytis cinerea at fixed relative humidity of 100% in a range of temperatures between 10 to 30 degrees Celsius for 9 hours.

FIG. 30 shows the graph of the spore germination rates of Botrytis cinerea at fixed relative humidity of 100% in a range of temperatures between 10 to 30 degrees Celsius for 9 hours.

FIG. 31 shows the spore germination rates of Botrytis cinerea at fixed temperature of 20 degree Celsius in a range of relative humidity between 70% and 100%.

FIG. 32 shows the summary of the validation results of the independent events of the general spore germination model.

FIG. 33 shows the photo of the spore germination experiments at the condition of 23 degrees Celsius and 97% relative humidity for 9 hours.

FIG. 34 shows the photo of the spore germination experiments at the condition of 13 degrees Celsius and 80% relative humidity for 9 hours.

FIG. 35 shows the main architecture of the IoT application of the occurrence prediction model.

### DETAILED DESCRIPTION OF THE EMBODIMENTS

The present disclosure is a framework under which systems and methods for predicting occurrences of different diseases and providing treatments thereof are developed. The framework makes use of machine learning and big data analysis, and includes a peptide prediction model and a disease occurrence prediction model.

Under the framework provided by the present disclosure, the peptide prediction model comprises a database involving an SCM-based antifungal peptide prediction system and related data of the target diseases. The disease occurrence prediction model is built by CNN technology to predict the probability and the outbreak timing of diseases. The components of the framework are connected by IoT technology, and the system works on cloud computing of aggregated data.

### Peptide Prediction Model

The peptide prediction model allows user to efficiently identify the target peptide for use as the control measure for a disease. To predict the target antifungal peptides for fungal diseases, an antifungal database is established with an antifungal prediction system to evaluate and predict for potential antifungal peptides and a search system containing relationships of hosts, pathogens, and corresponding peptides. Therefore, the antifungal database allows queries for the hosts, pathogens, and corresponding peptides according to the users' needs and potentiates its functions in both new drug discovery and old drug repurposing for the antifungal peptides.

The present disclosure utilizes artificial intelligence to strengthen the power of large datasets with an antifungal peptide prediction system, which is based on the SCM configured with further optimization. The antifungal peptide prediction system of the present disclosure evaluates and predicts the antifungal characteristic of a peptide based only on the sequence analysis, and provides a method for peptide prediction with simplicity, interpretability, and acceptable accuracy.

The SCM is based on Support Vector Machine (SVM), and is a method known in the literature [1]. To predict and evaluate the antifungal property of a peptide, SCM is introduced into the peptide prediction model with the perspective of biological information for machine learning. SCM used in the present peptide prediction model can not only predict the peptide function, but also the important domains of the peptides. In the present peptide prediction model, the SCM includes at least two parts, i.e., the calculation of the dipeptide score and the intelligent genetic algorithms (IGA) which is based on genetic algorithms.

The peptide prediction model is implemented with datasets, scoring of peptides by analyzing dipeptides and weights, and Intelligent Genetic Algorithm (IGA), which are further described herein.

Datasets of the peptide prediction model of the present disclosure comprises positive data and negative data. The positive data are the peptides that have antifungal properties and can comprise peptides from the antifungal databases, such as CAMP, PhytAMP, or those known in the literature and published in the public domain, such as PubMed. The negative data are the peptides that do not have antifungal properties and can comprise peptides that are not annotated as antifungal in the protein and peptide databases, for example, UniProt. Train dataset and test dataset are created by reducing the sequence identity of positive data and negative data, and the data are divided into two portions, so that each dataset has an equal amount of positive and negative data.

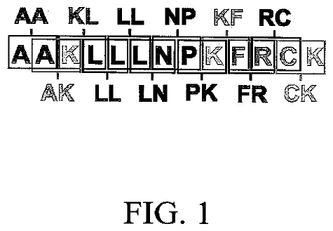

The “dipeptide” consists of two amino acids (AA) and is viewed as the smallest functional unit. FIG. 1 shows a peptide displayed as a group of dipeptides. The antifungal characteristic prediction of a peptide is based on the sequence analysis of the peptide. A peptide that has more potentially antifungal dipeptides will be more likely an antifungal peptide, and vice versa. The dipeptide propensities for the entire 400 individual dipeptides are obtained by statistical discrimination between dipeptide compositions of the antifungal peptides and non-antifungal peptides. Each dipeptide frequency of each peptide is to multiply a weight to get a score. If the score of the peptide is higher than the threshold tallied out, then it is predicted as an antifungal peptide. A higher score of the peptide indicates the higher probability of the antifungal function it possesses.

The initial weight value for each dipeptide is the ratio of the dipeptide appearing in the positive datasets minus the ratio appearing in the negative datasets. The weight value is then further optimized by IGA.

A selection method is used for selection of weight. Two weights are picked up among all: the one that had the highest fitness value, or the one selected by a selection method. The fitness value is calculated as a function of correlation coefficient between the initial and optimized propensity scores and AUCs, which are the Area under ROC (Receiver Operating Characteristic) Curves. An AUC closer to 1 indicates higher accuracy of the prediction model.

The peptide prediction model further comprises IGA (intelligent genetic algorithm), where crossover selection and optimization are implemented. Herein, crossover selection is a pair of parameters of the two weights that are randomly selected to exchange. Optimization is a known art [2] and involves a creative method for large parameters optimization in which the selection function has been designed to simplify the numbers of different parameter sets.

The peptide prediction model is further configured to comprise a search system containing connections between related data in the peptide prediction. The related data can include hosts, pathogens and peptides. These related data are aggregated into a single antifungal database which provides an efficient search for potential peptides for a given host or a given pathogen. The antifungal database also allows cross-match between hosts and peptides or between pathogens and peptides, thereby realizing repurposing of a previously identified drug.

### Disease Occurrence Model

The disease occurrence model provides daily possibility of disease occurrences. In the disease occurrence model, the Convolutional Neural Network (CNN) method is used to catch the weather patterns that was hard to be recognized by humans. Furthermore, the disease occurrence model is coupled with a warning system and an auto-spraying system with the IoT technology to apply the predicted peptide from the peptide prediction model into the farms.

The disease occurrence model is implemented with datasets that include the past fungal disease data and the weather data, based on the CNN method with a softmax function, a model cost function and an optimizer. Further, the disease occurrence model is connected to a system of IoT, which is further described herein.

The disease control system of the present disclosure is based at least in part the weather conditions that are shown to be related to fungal disease occurrence incidence. In the present disclosure, weather data presented by the 4 weather conditions, i.e., relative humidity, temperature, air pressure, and rainfall, are collected for over a period of time. In one embodiment, the weather data is collected for the past 14 days. In one embodiment, a total of 11 features based on the collected weather data are used in the convolutional neural network (CNN) to calculate the daily probability of the disease occurrence. In another embodiment, in addition to CNN, the spore germination rate is also calculated to provide a prediction of the accurate time of spore germination. The components of the system, such as the sensors for collecting the data and the sprayers that apply the predicted peptide based on the predicted occurrence time are connected over IoT.

The disease occurrence model comprises two different kinds of data, i.e., the fungal disease data and the weather data at the time when fungal diseases happened. The fungal disease data can be obtained from the government agency, and the weather data is collected from the Central Meteorological Bureau. Preprocessing of the data includes combining the fungal disease data and the weather data, and the fungal disease data that had no corresponding weather data are deleted. These data are then standardized for machine training and testing. CNN is adopted to recognize the patterns of the weather data features automatically. The pattern of the favorite weather change for the fungal diseases is recognized and caught by CNN. In the disease occurrence model of the present disclosure, CNN was used to identify the weather change that was suitable for occurrence of fungal diseases at a specific time.

The disease occurrence model further comprises a max pooling layer in addition to the CNN. After data went through the CNN layers, the amount of data increases enormously, and an added max pooling layer helps to reduce the computational complexity of the model and helps to find the best tendency of the data.

The disease occurrence model further comprises a full connection layer that converted the max pooling output into high dimensional space, and classified them into two classes, i.e., negative (diseases that had not happened) and positive (diseases that had happened).

The disease occurrence model further comprises a softmax function to transform the output from CNN into the disease occurrence probability. The network output before transformation can be hard to be realized by humans. The softmax function transforms the output into the disease occurrence probability that could be understood by both machines and humans.

In addition, cross-entropy is used to evaluate the differences between the predicted values and actual values in the training stage. Independent data used to test the disease occurrence model showed that the model delivers an accuracy of 83%.

The disease occurrence model further comprises spore germination modeling that predicts a spore germination rate, and hence the prediction of the occurrence of diseases is more effectively and timely. The spore germination modeling comprises fitting a linear equation for the spore germination rate based on the humidity and a cubic equation for the spore germination rate based on the temperature. A general spore germination modeling is thereby obtained by multiplying the two. The spore germination experiments are carried out to verify the modeling and determine the coefficients.

### EXAMPLES

In the following descriptions of the present disclosure, reference is made to the exemplary embodiments illustrating the principles of the present disclosure and how it is practiced. Other embodiments will be utilized to practice the present disclosure, and structural and functional changes will be made thereto without departing from the scope of the present disclosure.

### Example 1. Establishing of the Peptide Prediction Model

Collection of positive (antifungal) dataset was obtained from online public databases such as CAMP, APD, PhytAMP, in addition to the new peptides that are collected in the local database, while the negative (peptides without antifungal property) dataset was collected from the public database for proteins and peptides, UniProt, where they do not have antifungal or antimicrobial characteristics associated.

The collected datasets undergo pre-processing including deleting the peptides that contain non-standard amino acids. Then, the peptides of the datasets are limited to lengths of between 10 AA's to 100 AA's because antifungal peptides are typically between 10 and 100 amino acids long. Furthermore, the peptides are filtered with identity of no more than 25%. Then, equal amount of negative data and positive data are selected. Afterwards, the positive and negative dataset are randomly distributed, and one-third of the data is used as an independent testing set.

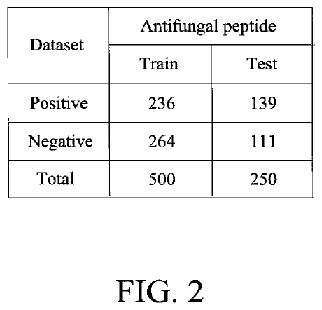

FIG. 2 shows the number of the datasets collected and used, with a total of 375 positive and 375 negative data, and two-thirds of the dataset are randomly selected to act as the training data, while one-third of the dataset is the independent testing dataset.

Briefly, for every peptide in the dataset, the dipeptide frequency is calculated. Then, an initial weight for each specific dipeptide is given through statistical methods. Multiplying the dipeptide frequency matrix by the weight matrix tallied out the peptide score. For a peptide evaluated, the higher score it is, the greater possibility it possesses an antifungal function.

### Dipeptide Frequency

Each peptide will form a 400×1 matrix of dipeptide frequency because there are 20 types of amino acids (AA) which result in 400 types of dipeptide frequencies as shown by the formula below:

20_{AA}×20_{AA}=400_{dipeptide}

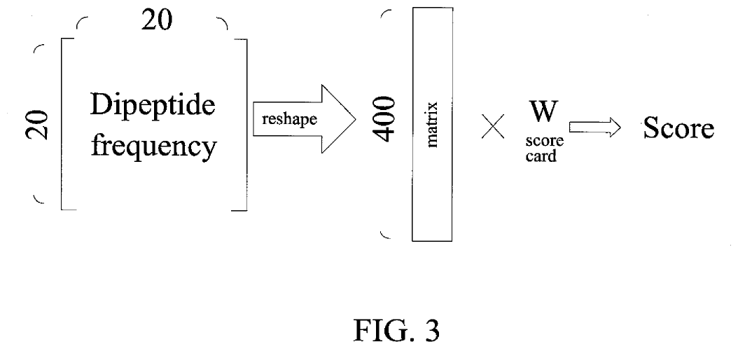

Each peptide will then get a score according to its peptide sequences by multiplying the dipeptide frequencies with the scorecard matrix of weight. FIG. 3 illustrates how to calculate a peptide score by the scorecard. First, the 20×20 matrix is reshaped into the 400×1 matrix, and then multiplied with the scorecard matrix A final score is obtained thereafter by the formula below, where xᵢ is the dipeptide frequency and wᵢ is the corresponding weight:

$${\sum }_{i = 0}^{400} {x}_{i} \cdot {w}_{i} = score$$

The calculated score of the peptide compared with the threshold to predict its propensity as an antifungal peptide or a non-antifungal peptide.

$$f ( x ) = \{ \begin{matrix}if x > threshold \text{:} {f}_{( x )} = positive \\ if x \leq threshold \text{:} {f}_{( x )} = negative\end{matrix}$$

### Weight

The initial weights used in the scoring of the peptide include first determining P(ij), the dipeptide frequency of positive dataset, and N(ij), the dipeptide frequency of negative dataset, which are calculated by the equations below, where nᵢⱼ and Lₚ₋₁ represent number of occurrence of the ij^{th} dipeptide and the sum of the lengths of all peptides each minus 1, respectively:

$$P ( ij ) = ( \frac{{n}_{ij}}{{L}_{p - 1}} \vert 𝒞 = 1 ) , 1 \leq i , j \leq 20$$

$$N ( ij ) = ( \frac{{n}_{ij}}{{L}_{p - 1}} \vert 𝒞 = 0 ) , 1 \leq i , j \leq 20$$

Then, each weight (S(ij)) is obtained from the calculation that the frequency of positive data (P(ij)) minus the frequency of negative data (N(ij)):

Sᵢⱼ=Pᵢⱼ−Nᵢⱼ

The individual weight thus obtained is normalized to [0,1] and then times 1000:

$${S}_{( ij )}^{′} = ( \frac{{S}_{ij} - {S}_{min}}{{S}_{max} - {S}_{min}} ) \times 1000$$

The initial scoring card containing a set of dipeptide weights S′₍ᵢⱼ₎ is thus obtained.

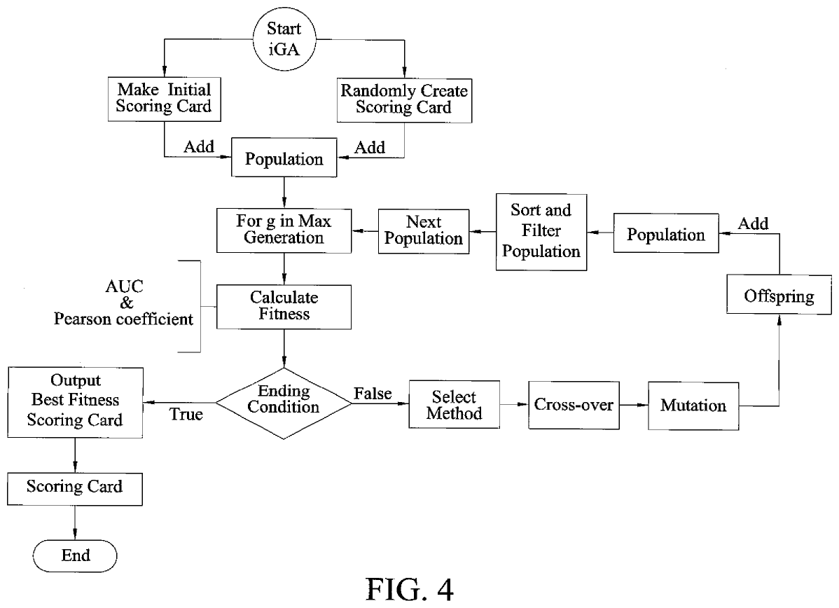

IGA is then used to optimize the initial scoring card. FIG. 4 shows the flow chart of IGA implementation. First, the initial scoring card with another randomly initialized scoring card is combined to make the first population. Then, the fitness of every scoring card is calculated. If the scoring cards reach the ending condition, it will return the final score card with the best fitness in training data. To get the best accuracy and prevent the model from over-training, the ending condition of the program is to be terminated after 30 generations. If the ending condition is not yet met, the scoring card is switched to the selection section to select many pairs of scoring cards into the crossover section to make new offspring scoring cards. The new offspring scoring cards are then passed to the mutation section. After the mutation, the new offspring would be added into the population, and the population would be ranked by their fitness. Further, the scoring card that ranks out of the max_population would be removed.

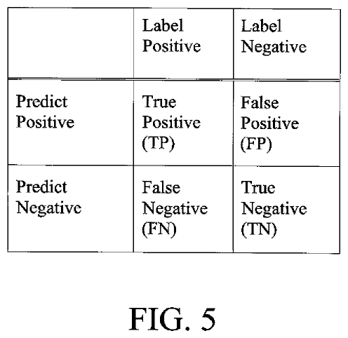

The fitness calculation is further described herein. First, confusion matrix is calculated by separating into a prediction section and a label section and categorizing into four classes: TP (True Positive), FP (False Positive), FN (False Negative), and TN (True Negative), as shown in FIG. 5. Then, true positive ratio (TPR) and false positive ratio (FPR) are calculated as follow:

$$TPR = \frac{TP}{( TP + FN )}$$

$$FPR = \frac{FP}{( FP + TN )}$$

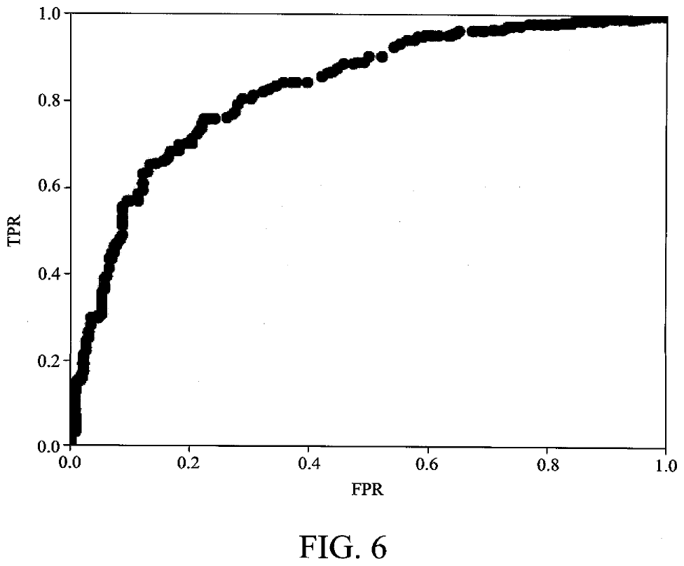

Then, the ROC curve is drawn taking TPR as the y-axis and FPR as x-axis, as shown in FIG. 6. With different thresholds to distinguish positive data from negative data, the TP, FP, FN, and TN would be different. As a result, different TPR and FPR for each threshold are obtained, and therefore, the ROC curve is drawn with each TPR and FPR. To evaluate the fitness of the weight, the Area Under the ROC Curve (AUC) is calculated. The AUC of the ROC curve suits for models with unbalancing datasets, such as in the present example where non-antifungal peptides are far more than antifungal peptides.

In addition to the AUC value, the Pearson coefficient of the amino acids between the initial scoring card and the scoring card under test is also considered for fitness calculation. Different weights are given for each value, with 0.9 of the AUC value and 0.1 of the Pearson coefficients for the best training performance. Use of the Pearson coefficient in the model avoids overtraining.

Then, to optimize the initial scoring card, advanced crossover is used to produce variation for machine learning. For each round, two weights are selected by a selection method. After the advanced crossover is optimized from the normal crossover, mutation is done, and new weights are put into the population.

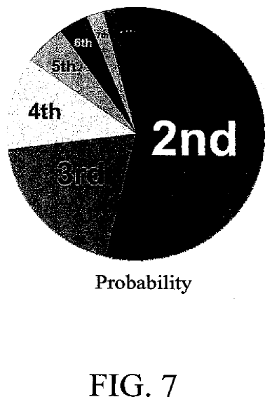

Specifically, the selection method involves picking two weights from all weights, with one having the most fitness value, namely the highest AUC, which is probably the best weight, and the other weight being selected using the roulette method. The roulette method is done by separating each weight of the score card into different areas in proportion to its fitness. The higher fitness of the weight would get the larger area (FIG. 7). Then, a number was randomly chosen, and a score card was selected from the area of the random number. The roulette method is used to ensure the randomness of the selection. Thus, the score card with higher fitness will probably be chosen but not absolutely.

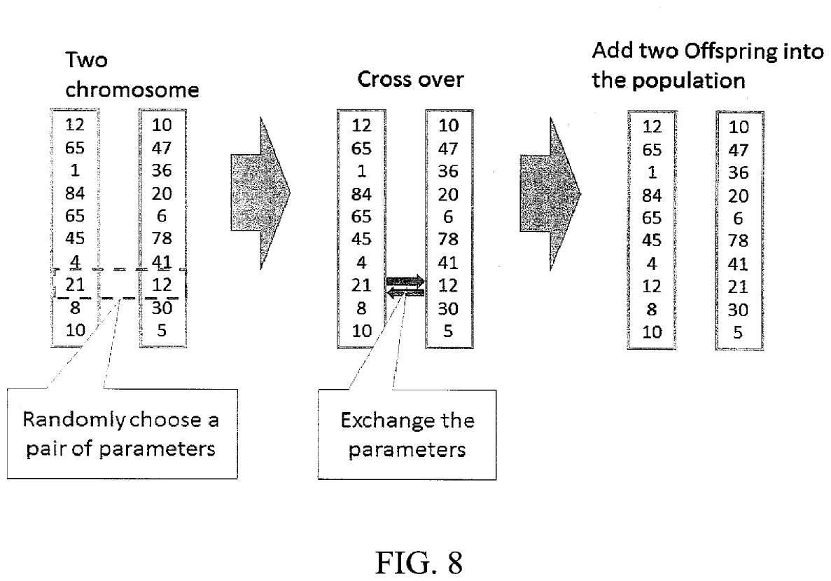

After the parents are selected, IGA is used to optimize the crossover. IGA is based on the normal Genetic Algorithms (GA) where the crossover selection is the most important selection. After selecting two parents, crossover involves choosing a pair of parameters to exchange, and then the exchanged score card is returned into the new population (FIG. 8). Then, the lower fitness score card is deleted to keep the population in a range.

To choose the best set of parameters for crossover, a target function shown as follows is maximized, where each x₁, x₂, x₃ represents a pair of dipeptide frequencies under evaluation:

f(x₁,x₂,x₃)=100x₁−10x₂x₃

For each x₁, x₂, x₃, two candidates are chosen, just like the two parents in the crossover step. To maximize the function of IGA, the first step is to create an OA-array shown below:

maximize y(x₁,x₂,x₃)=100x₁−10x₂−x₃,

x₁∈{1,2},x₂∈{3,4}, and x₃∈{5,6}

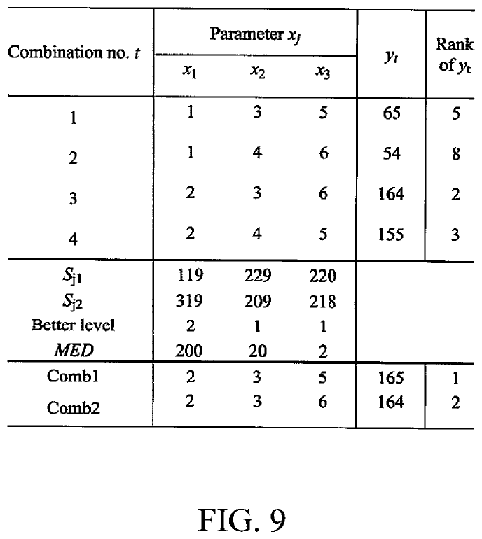

For evaluating the value of x₁, the key is to eliminate the effect by x₂ and x₃. FIG. 9 is an example for determining x₁ where it could be seen that to obtain the evaluation of x₁, combinations 1 and 2 are paired together, while combinations 3 and 4 are paired together. Because the value of the weight Sⱼ₂ is larger than of the weight Sⱼ₁, the better parameter for x₁ will be 2 instead of 1. The other parameters are chosen similarly. If the number of parameters is big enough, the effect of other parameters will be limited.

After the crossover, the new offspring undergoes a mutation section. In the mutation section, the program chooses a random number to determine whether to mutate or not. If the result is yes, it randomly chooses an allele of the offspring and sets a random number. The mutation section increases the randomness of the model.

Following the mutation section, the new offspring joins the population, and then the program sorts all scoring cards in the population according to their fitness values. After sorting the population, the last process was to filter out the scoring card that ranks outside the max_population number.

The program is terminated after 30 generations to avoid over training. When it reaches its end condition of 30 generations, it returns the final score card with the best fitness in training data.

### Example 2. Antifungal Peptide Prediction with an Antifungal Peptide Having Sequence Identity of 25%

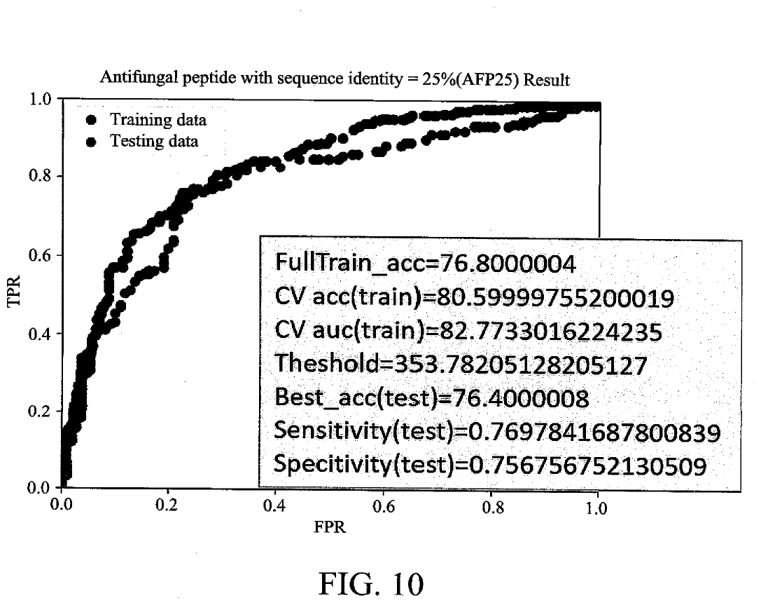

Following the steps described in Example 1, the final ROC curve and the result of test datasets with an antifungal peptide having sequence identity of 25% (AFP25) is shown in FIG. 10

The test accuracy, i.e., the overall performance of classifying positive data as positive and negative data as negative, is 76%. The sensitivity, i.e., the performance of classifying positive data as positive, is 77%. The specificity, i.e., the performance of classifying negative data as negative, is 76%. The suitable threshold value is 354, and peptide scores higher than this value is considered as an antifungal peptide.

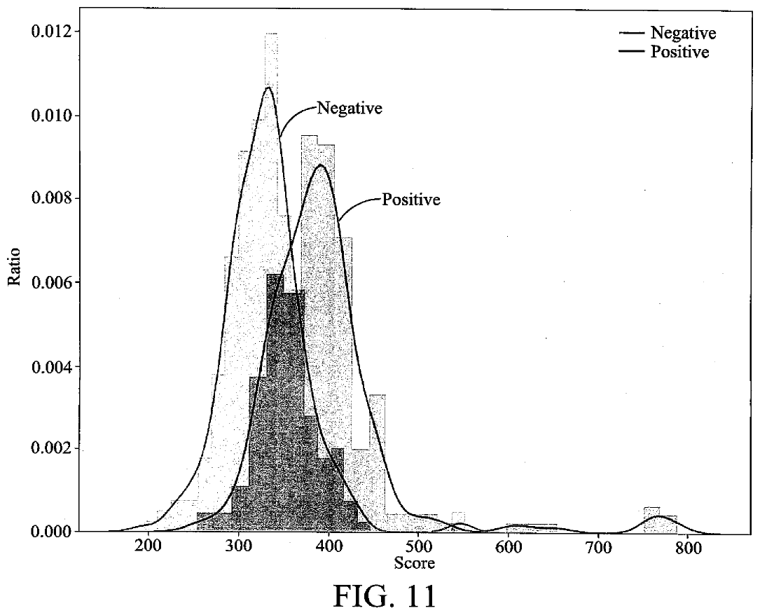

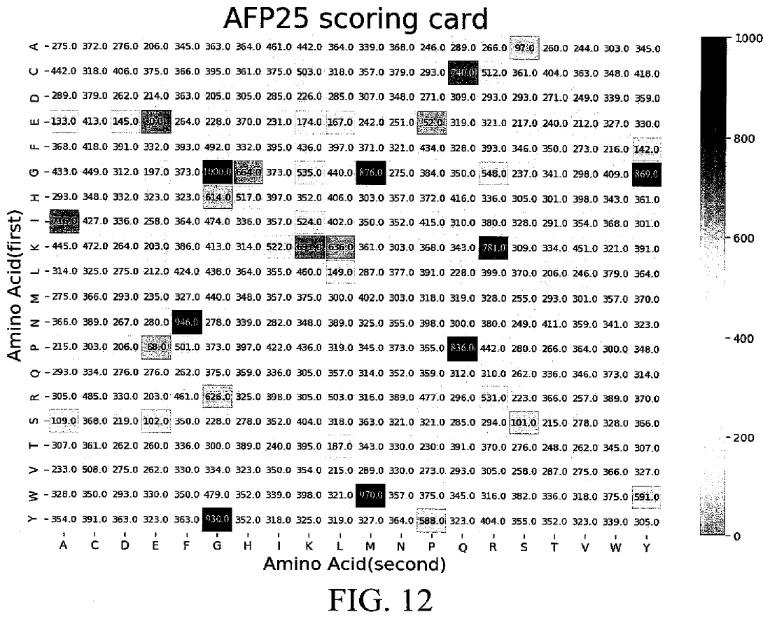

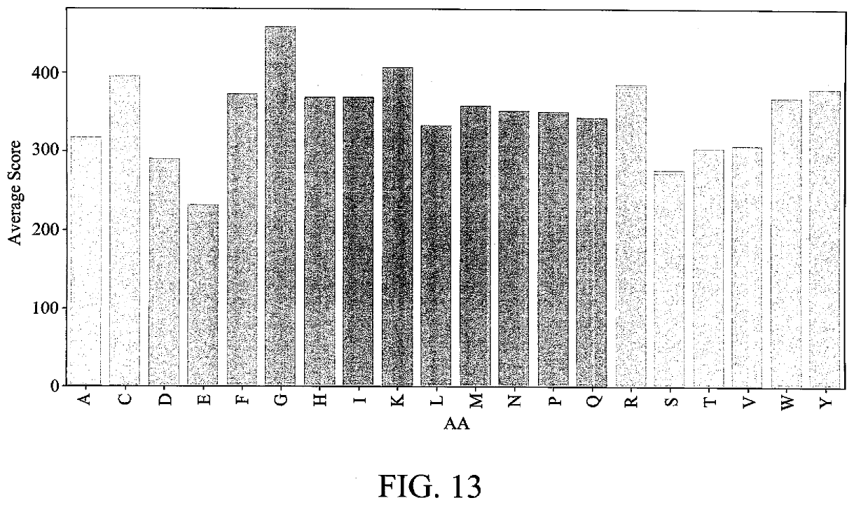

The score distributions of the positive datasets and negative datasets are shown in FIG. 11, and the final antifungal scoring card of the dipeptide scores is shown in FIG. 12. The single amino acid score calculated from each dipeptide score is shown in FIG. 13. From the score results, the top three amino acids are cysteine (C), glycine (G), and lysine (K), and the five amino acids to have lowest scores are aspartic acid (D), glutamic acid (E), serine (S), threonine (T), and valine (V). Many antifungal peptides for plants and mammals contain lots of cysteine, such as thionins, plant defensins, etc. There are also many glycine-rich peptides from insect's antifungal peptides.

For the five amino acids with the lowest scores (D, E, S, T, V), four of them are hydrophilic, while most of the hydrophilic amino acids have a higher score (average score=362.73>threshold: 350). Additionally, for the top 5 highest amino acids, cysteine contains a sulfide functional group that can form a disulfide bond, and lysine (K) and arginine (R) are easy to form a hydrogen bond.

### Example 3. Identification of the Active Site from Predicted Antifungal Peptide

To show the result of the scoring card, the peptides are visualized by color representation of the dipeptide score on its 3D structure. The region of a peptide with a higher dipeptide score is shaded darker. The region of a peptide with lower dipeptide score is represented with lighter shades. The important region of an antifungal peptide is thus identified by the dark-shaded area.

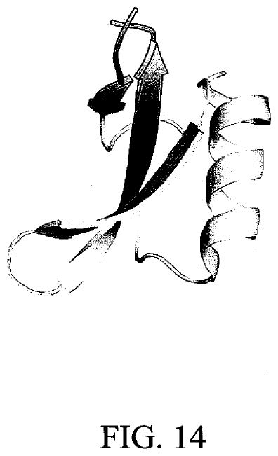

FIG. 14 shows the shaded 3D structure of Rs-AFP2 according to the dipeptide scores calculated by the prediction model, which is an antifungal peptide from the plant defensin family, where the N terminal of the peptide and the three beta sheets are the darkest shaded parts of the peptide. According to the scoring system based on the SCM, it indicates that these two regions are the regions that determine whether the whole peptide sequence is an antifungal peptide or not.

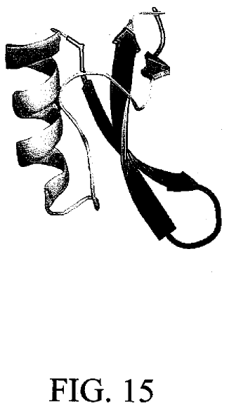

FIG. 15 shows the 3D structure of Rs-AFP2 peptide with its active region shaded darker according to the report by Schaaper [3]. According to this report, the major active sites are between the β2 and β3 loop, from Ala³¹ to Phe⁴⁹, and some activities are also found in the N-terminal part of the protein.

Therefore, the predicted active sites visualized in the 3D structure with scoring cards corresponds to that reported in the literature, indicating the SCM of the antifungal peptide prediction model indeed possesses the ability to correctly determine the antifungal active sites.

### Example 4. Modeling and Predicting Disease Occurrence

A model predicting the disease occurrence relating to the daily weather was established based on the neural network. There are two kinds of data used in the prediction system, i.e., the disease data collected from the government agency and the weather data from the Central Meteorological Bureau's website that correspond to the disease data. Then, the two data are combined, and the disease data that do not have the weather data to match with are deleted.

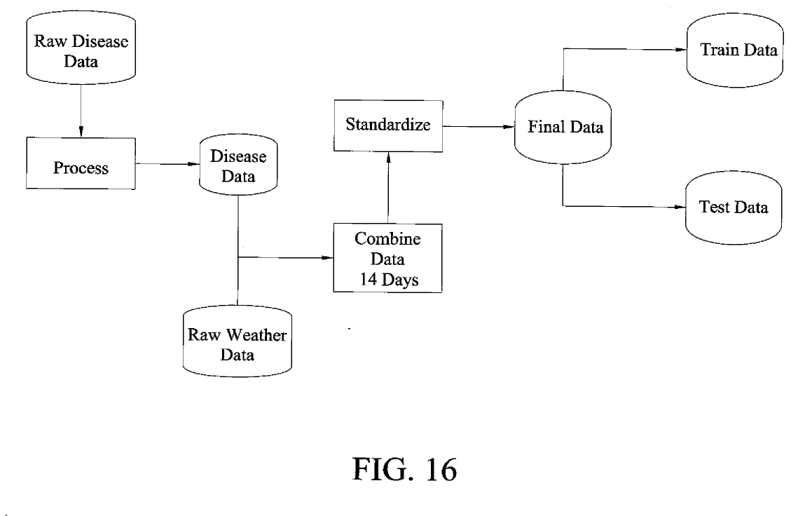

The final data then contains a weather feature and a label. The weather feature is a two-dimensional array having 14 days×11 features. The 11 features include relative humidity, rainfall and the maximum, minimum, average of the temperature and air pressure. The label contains two classes, negative (no disease occurrence) and positive (disease occurrence). The flow chart of data processing in the model is shown in FIG. 16.

The weather condition affects the spore germination and the health of plants. This relationship between the weather condition and the disease occurrence is recognized by the Convolutional Neural Network (CNN) to catch the specific weather patterns that lead to disease occurrences.

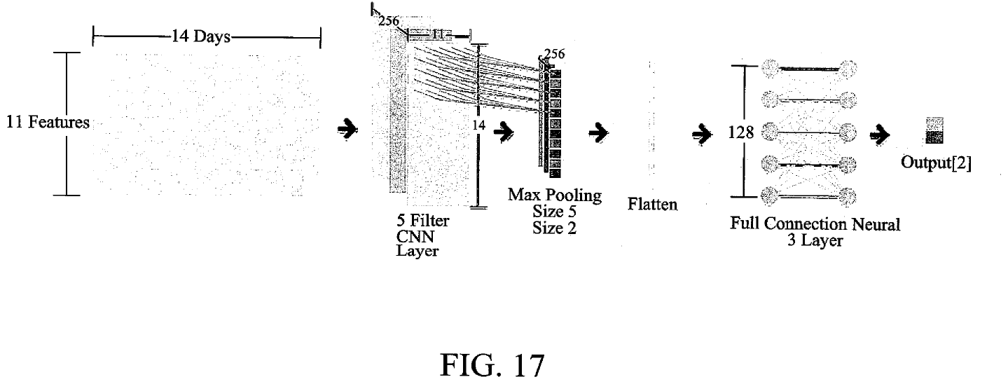

FIG. 17 shows an overview of the CNN method used in the model, where it contains the convolution layer, max pooling layer and multi full connection layer. The model uses the weather data for the past two weeks as the model input, and the weather patterns are recognized from this 14-day weather data. After the CNN layer, the weather features are converted to weather change features, and then a max pooling layer is added to filter noises after the CNN layer. The weather patterns that cause diseases do not change in a short time, so that the function of the max pooling is to only return the maximum values in the filter.

f(xᵢ)=max(xᵢ)

For example, if the input array is [2,5,1,7,0,4] and the max pooling filter size is 2, when the filter step is 1, which is the distance the filter moves, the first max pooling output will be max(2,5)=5, and the second output is max(5,1)=5 and so on. Because the max pooling output is a two-dimensional tensor, the max pooling output is flattened to one dimensional tensor for a further full connection layer.

After flattening the max pooling layer, the full connection layer is used to classify the max pooling result. The full connection layer is a basic neural network layer that can switch the max pooling layer output into the high dimensional space and then classify them into two classes, namely the negative (no disease occurrence) and positive (disease occurrence).

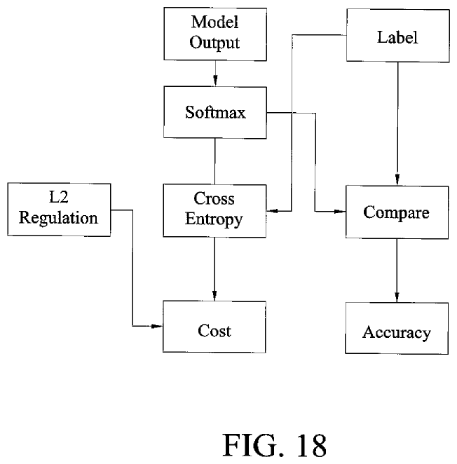

However, the network output is a number that is difficult for humans to understand and use, so that the softmax function is used to transform the number into the disease occurrence probability (FIG. 18). The following is the formula of the softmax function used:

$${σ ( z )}_{j} = \frac{{e}^{{z}_{j}}}{{\sum }_{k = 1}^{K} {e}^{{z}_{k}}}$$

In the above formula, σ is softmax function, Z is final output of the network, K is the total number of outputs, and thej is the j^{th} output.

Afterwards, cross-entropy is chosen as the network cost function because it performs well in the exclusion classification mission. The formula used for the cross-entropy is as follows:

$${H}_{y} ′ ( y ) = - {\sum }_{i} {y}_{i}^{′} log ( {y}_{i} )$$

In the above formula, H is a cross-entropy function, y′ᵢ is the real label, and the y is the network prediction output.

Parameters of the neural network are then optimized by an Adam optimizer, which is the most commonly used way to optimize the network.

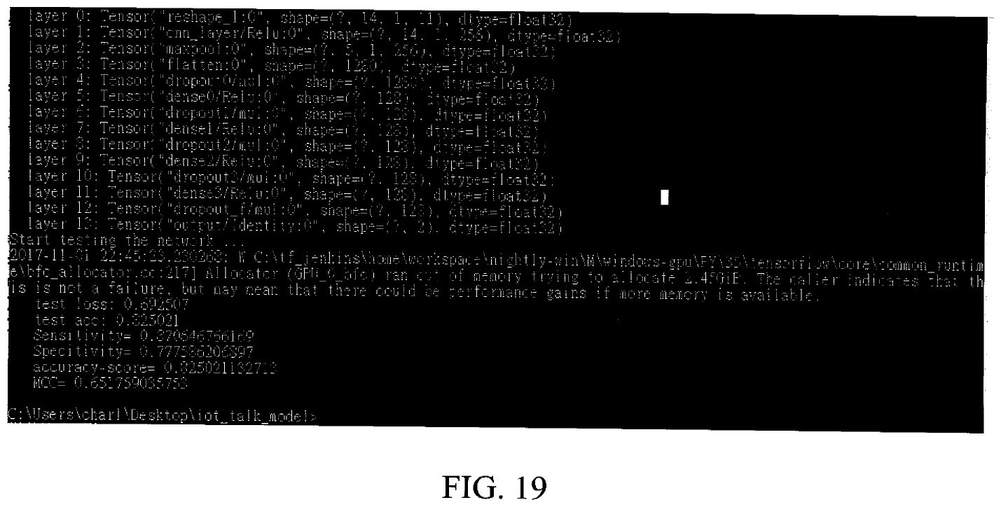

After training, the model is tested by an independent test data with the result shown in FIG. 19, where the accuracy score is as high as 82.5%.

### Example 5. Modeling and Prediction of Spore Germination Rate

In addition to the daily disease occurrence prediction with the CNN method described in Example 4, a more accurate timing of the prediction is made by including the calculation of the spore germination rate, since the spore germination must happen before disease occurrence. The conditions that lead to spore germination are identified and confirmed by experiment.

Humidity and temperature are found to affect the spore germination the most. A general model for the spore germination rate based on temperature or humidity is built with different fungal species which also fits for every fungal species.

First, the spore germination data published in the literature are used to fit out the functions. As a result, the spore germination rate based on temperature is fitted by a cubic equation: f₁(x)=ax³+bx²+cx+d, where x represents the temperature. The spore germination rate based on humidity is fitted with a linear equation: f₂(x)=ax+b, where x represents the humidity. The general spore germination rate is therefore f₁(x)×f₂(x).

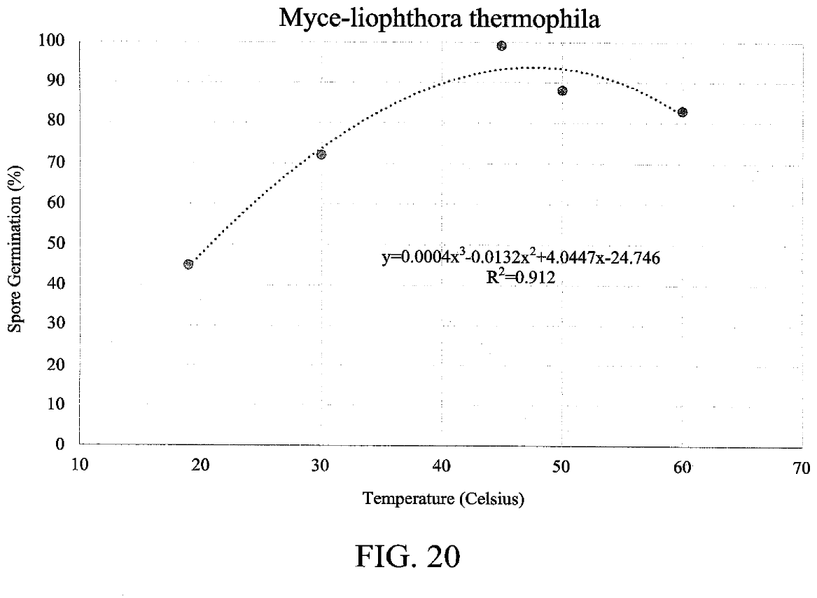

Myce-liophthora thermophila's spore germination rate based on temperature is shown in FIG. 20 and the equation fitted is:

y=0.0004x³−0.0132x²+4.0447x−24.746

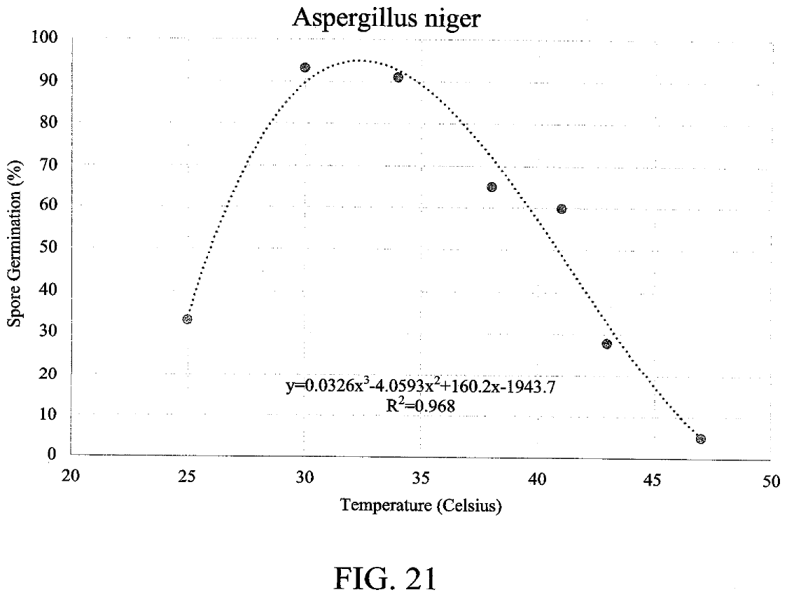

Aspergillus niger's spore germination rate based on temperature is shown in FIG. 21 and the equation fitted is:

y=0.0326x³−4.0593x²+160.2x−1943.7

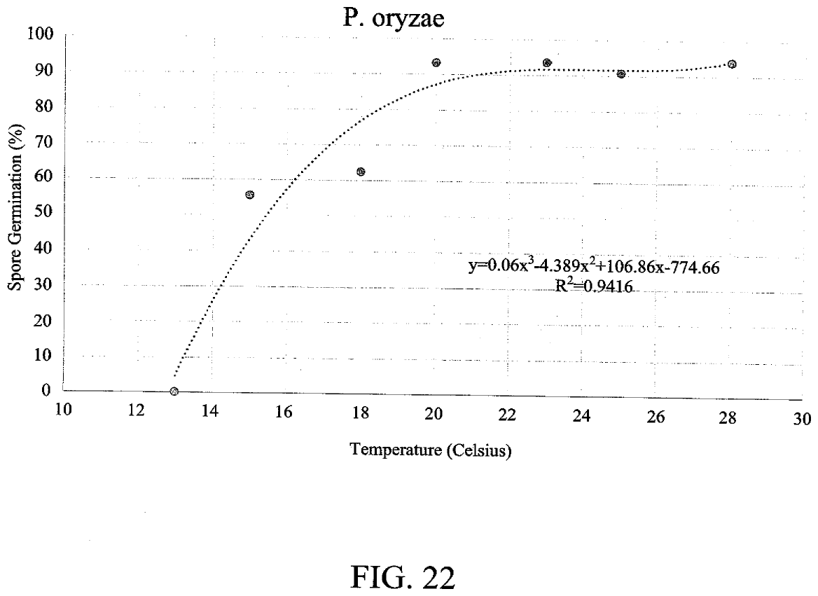

P. oryzae's spore germination rate based on temperature is shown in FIG. 22 and the equation fitted is:

y=0.06x³−4.389x²+106.86x−774.66

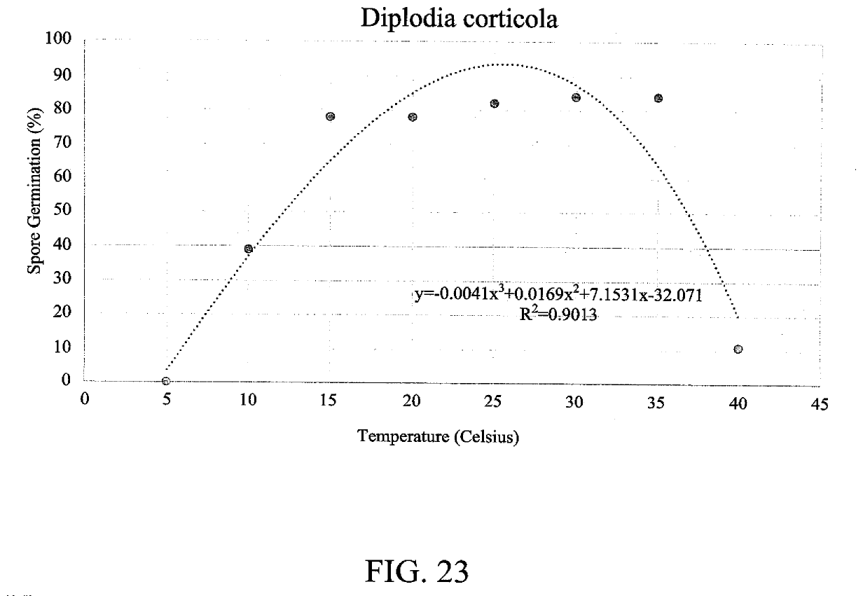

Diplodia corticola's spore germination rate based on temperature is shown in FIG. 23 and the equation fitted is:

y=−0.0041x³+0.0169x²+7.1531x−32.071

Therefore, the spore germination rate based on temperature is fitted in a cubic equation:

f₁(x)=ax³+bx²+cx+d

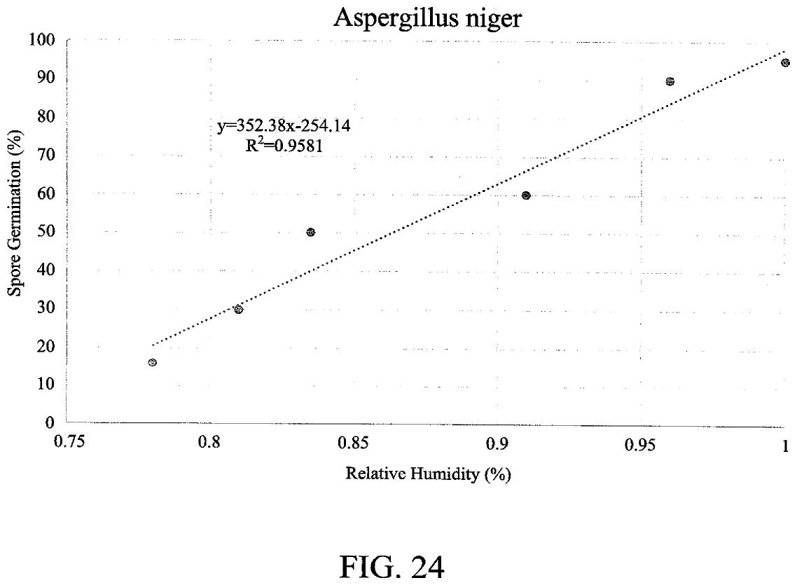

For the spore germination rate based on humidity, Aspergillus niger's spore germination rate based on relative humidity is shown in FIG. 24 and the equation fitted is:

y=352.38x−254.14

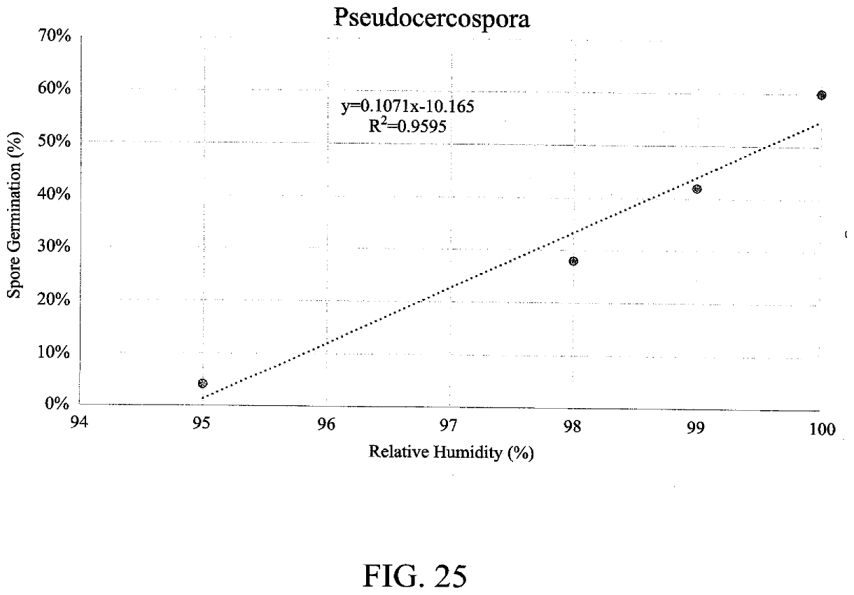

Pseudocercospora's spore germination rate based on relative humidity is shown in FIG. 25 and the equation fitted is:

y=0.1071x−10.165

As results, the spore germination rate based on the relative humidity is a linear equation:

f₂=ax+b

The two equations are considered as independent events and are multiplied to form the general fungal spore germination model:

f₁×f₂

Therefore, temperature and humidity conditions are the only factors needed to calculate the spore germination rate in the specified environment.

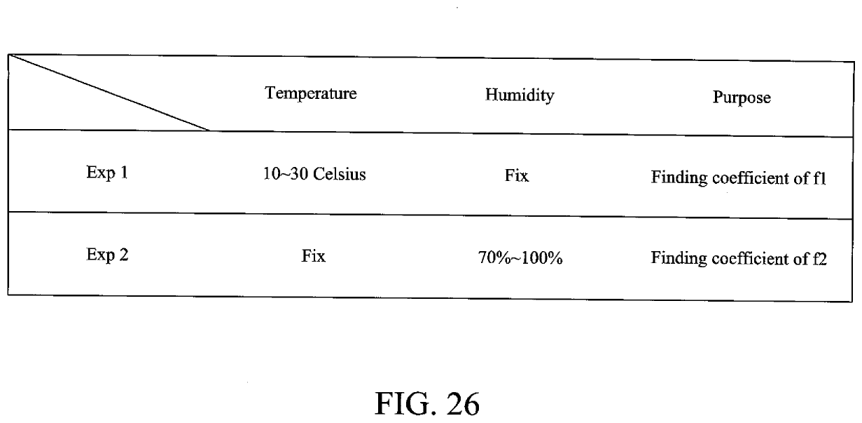

Experiments are then conducted to determine the coefficients for the general fungal spore germination model and to verify the model. The experiment was divided into two parts, as shown in FIG. 26. First, the temperature is fixed under varied humidity conditions by mixing equal volumes of the spore suspension solution (2×10⁵ particles/mL), which is made by removing the spores from a fungal plate by distilled H₂O, and 2% glucose solution in a concave glass slide placed in the temperature and humidity control box. The temperature was fixed at 25 degree Celsius, and the humidity conditions tested are in 5% increments from 80% to 100%. Then, the humidity is fixed under varied temperatures by mixing equal volumes of the spore suspension solution (2×10⁵ particles/mL) and 2% glucose solution in the concave glass slide placed in the temperature and humidity control box. The humidity was fixed at 100%, and the temperatures tested range from 10 to 30 degree Celsius in 5 degrees increments.

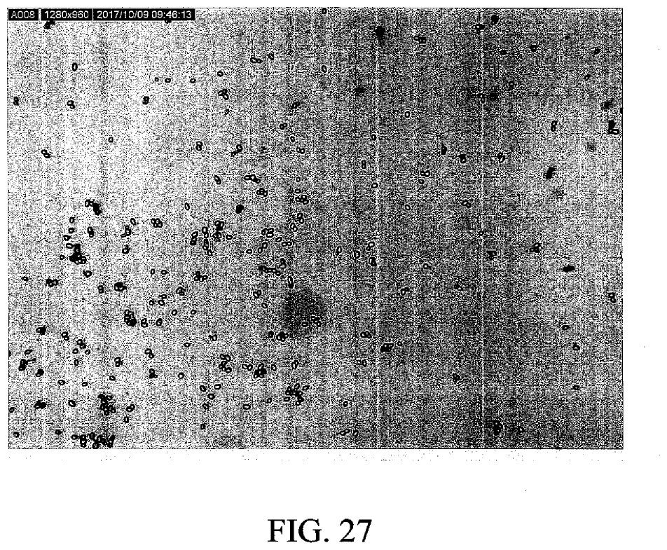

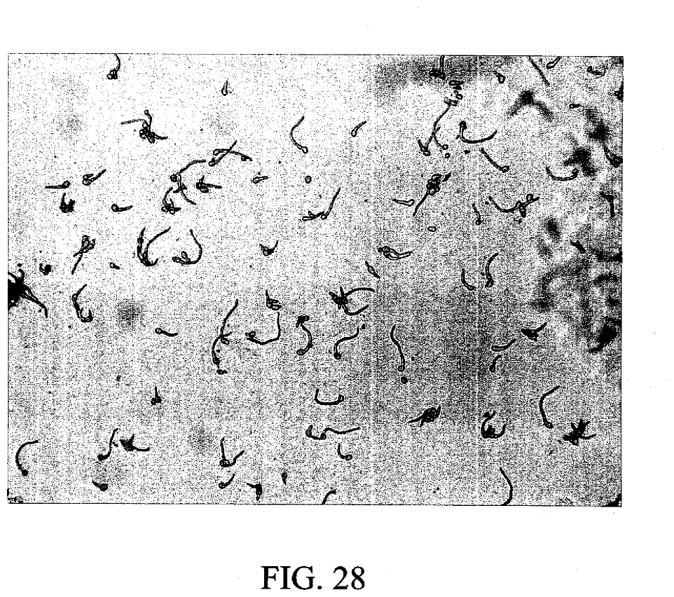

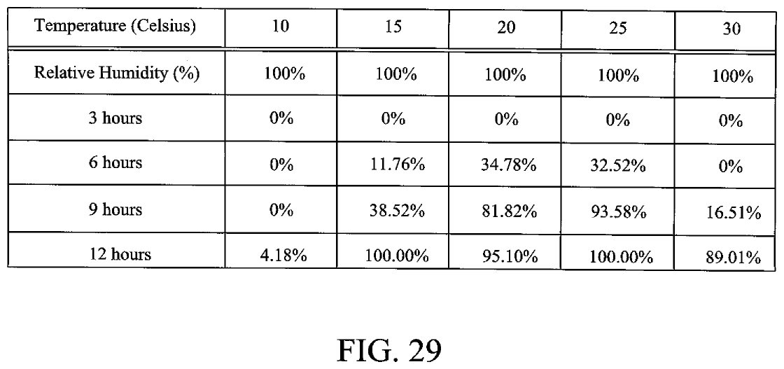

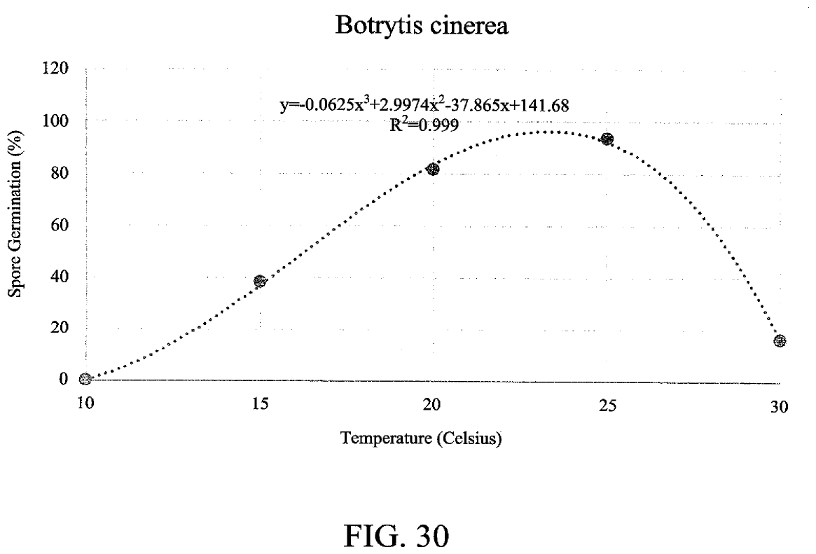

FIG. 27 shows the spores that had not germinated at 10 degree Celsius and 100% relative humidity for 9 hours. FIG. 28 shows the germinated spores at 25 degrees Celsius and 100% relative humidity for 9 hours. FIG. 29 shows the table of spore germination rates of Botrytis cinereal at fixed relative humidity of 100% in a range of temperatures between 10 to 30 degrees Celsius for 9 hours each, and FIG. 30 show the curve plotted based on the germination rates results.

The Botrytis cinerea's spore germination rate based on temperature is thereby:

f₁=(−0.0625x³+2.9974x²−37.865x+141.68)

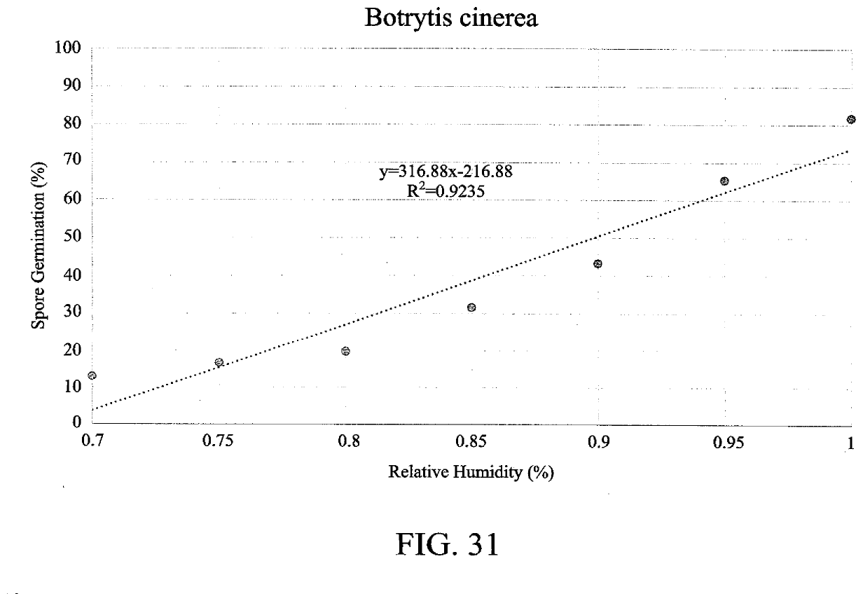

FIG. 31 shows the spore germination rates of Botrytis cinerea at fixed temperature of 20 degree Celsius in the range of relative humidity between 70% and 100%. Linear translation is used to make the spore germination 100% under 100% relative humidity.

The Botrytis cinerea's spore germination rate based on relative humidity is thereby:

f₂=316.88x−216.88

The result of the formula derived from the above is compared with the value of the spore germination in the reality to verify that the temperature and relative humidity are independent events to spore germination. The conditions are randomly chosen to be at temperatures of 23 and 13 degree Celsius, and in the relative humidity of 97% and 80%.

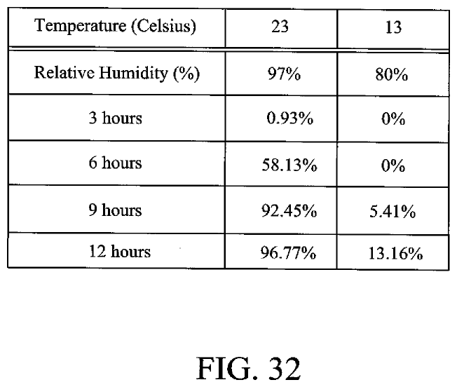

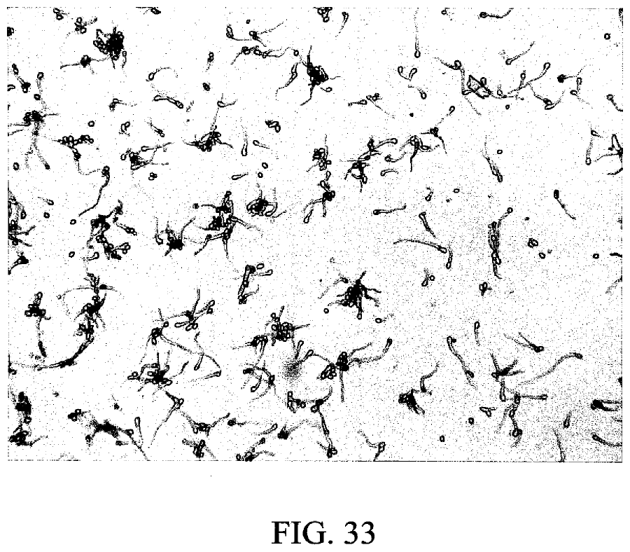

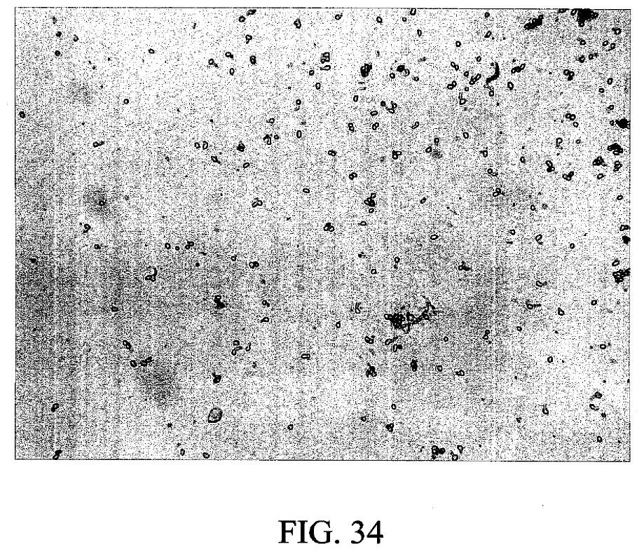

FIG. 32 shows the summary of the validation results of the independent events. At the condition of 23 degrees Celsius and 97% relative humidity (9 hours), the spore germination rate by experiment is 92.45%, and the spore germination calculated according to our formula is 86.84%. (FIG. 33) The spore germination under the condition of 13 degrees Celsius and 80% relative humidity (9 hours) by experiment is 5.41%, and that calculated according to our formula is 6.84%. (FIG. 34)

Therefore, the final formula of the spore germination model is:

f₁×f₂=[(−0.0625x₁³+2.9974x₁²−37.865x₁+141.68)÷100]×[(316.88x₂−216.88)÷100]

In the formula, x₁ is temperature, and x₂ is relative humidity. According to the above experiment results, the temperature and relative humidity are proved to be independent events, and the formula for the model is quite precise.

### Example 5. Application of the Disease Occurrence Prediction Model Over IoT

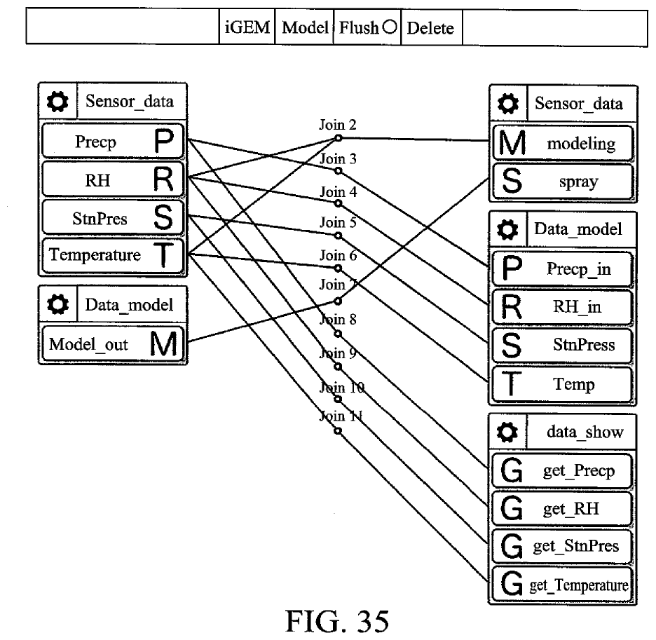

Sensors of the weather conditions such as those detecting temperature and humidity are connected over IoT and transfer the values to the processors of the prediction model. The daily probability of disease occurrence is calculated. If the calculated probability exceeds a certain value, which can be set by the user, the user is informed that the disease may occur, and advised to spray the predicted antifungal peptide. The user is allowed to decide whether to automatically spray. FIG. 35 shows the main architecture of the IoT application.

While some of the embodiments of the present disclosure have been described in detail in the above, it is, however, possible for those of ordinary skill in the art to make various modifications and changes to the particular embodiments shown without substantially departing from the teaching and advantages of the present disclosure. Such modifications and changes are encompassed in the spirit and scope of the present disclosure as set forth in the appended claims.

The references listed below cited in the application are each incorporated by reference as if they were incorporated individually.

### REFERENCES

[1] Huang, H.-L., Charoenkwan, P., Kao, T.-F., Lee, Chang, F.-L., Huang, W.-L., Ho, S.-Y., Shu, L.-S., Chen, W.-L., and Ho, S.-Y. “Prediction and analysis of protein solubility using a novel scoring card method with dipeptide composition.” BMC Bioinformatics, 13 (Suppl. 17), S3 (2012).
[2] Shinn-Ying Ho, Li-Sun Shu and Jian-Hung Chen, “Intelligent evolutionary algorithms for large parameter optimization problems,” in IEEE Transactions on Evolutionary Computation, Vol. 8, No. 6, pp. 522-541, December 2004 doi. 10.1109/TEVC.2004.835176.
[3] W. M. M. Schaaper, G. A. Posthuma, R. H. Meloen, H. H. Plasman, L. Sijtsma, A. Van Amerongen, F. Fant, F. A. M. Borremans, K. Thevissen, and W. F. Broekaert, “Synthetic peptides derived from the β2-β3 loop of Raphanus sativus antifungal protein 2 that mimic the active site.” Chemical Biology & Drug Design, Vol. 57, Issue 5, pp. 409-418 (2002).

1. A method for disease control, comprising:
disposing a plurality of sensors to detect environmental information to acquire weather data;
installing a processor to build up a disease prediction model;
collecting and combining disease data and the weather data, and simultaneously using the disease data and the weather data to calculate a spore germination model so as to indicate a plurality of patterns probability of diseases via Convolutional Neural Network (CNN) through the processor; and
wherein the weather data includes at least one of observation time, pressure, temperature, dew point temperature, relative humidity, wind speed, wind direction, precipitation, sunshine duration, visibility, ultraviolet index, and cloud amount over a period of time.

2. (canceled)

3. (canceled)

4. The method of claim 1, wherein the processor is configured to build up the disease prediction model further by: extracting features from the disease data and the weather data, and wherein the features are processed by the processor for the machine training and testing process.

5. (canceled)

6. The method of claim 1, wherein the processor is configured to build up the disease prediction model further by: classifying the patterns into a negative output indicating a disease not to be happened or a positive output indicating a disease to be happened.

7. The method of claim 6, wherein the disease prediction model is configured to raise warning according to the positive output.

8. (canceled)

9. (canceled)

10. The method of claim 1, wherein the processor is further configured to build up a spore germination model by fitting a linear equation for a spore germination rate based on humidity and a cubic equation for the spore germination rate based on temperature.

11. (canceled)

12. (canceled)

13. The method of claim 1, wherein the spore germination rate is represented by a formula below:
f₁×f₂=[(−0.0625x₁³+2.9974x₁²−37.865x₁+141.68)÷100]×[(316.88x₂−216.88)÷100],
wherein x₁ refers to the temperature in Celsius and x₂ refers to the relative humidity.

14. (canceled)

15. (canceled)

16. (canceled)

17. (canceled)

18. (canceled)

19. (canceled)

20. The method of claim 1, further comprising a step of connecting to a spraying system configured to spray a peptide with an antifungal function on a field based on the probability of the disease occurrence.
